(19) United States
(12) Patent Application Publication
(10) Pub. No.: US 2013/0238002 A1
(43) Pub. Date: Sep. 12, 2013

(54) VEIN DISRUPTION DEVICE

(71) Applicants: Edward M. Boyle, Bend, OR (US); Chris Genau, Seattle, WA (US); Andrew Jones, Bend, OR (US)

(72) Inventors: Edward M. Boyle, Bend, OR (US); Chris Genau, Seattle, WA (US); Andrew Jones, Bend, OR (US)

(21) Appl. No.: 13/469,278

(22) Filed: May 11, 2012

Related U.S. Application Data
(60) Provisional application No. 61/485,186, filed on May 12, 2011.

(51) Int. Cl. A61B 17/3205

(52) U.S. Cl. CPC A61B 17/3205
USPC 606/159

(57) ABSTRACT

A medical device for percutaneous disruption and therefore treatment of veins.

### CROSS REFERENCE

This filing claims the benefit of U.S. patent application Ser. No. 61/485,186, filed May 12, 2012, and also relates to U.S. patent application Ser. No. 12/766,165, attorney docket 4495, entitled Vein Removal Device, filed Apr. 23, 2010, and co-owned and sharing at least one inventor.

### BACKGROUND

The use of needle like devices to intercept and destroy vein is well known from U.S. Pat. No. 6,224,618 to Gordon among others. Although this therapy is well accepted there is a continuing need to make improved devices, which can quickly accurately and safely disrupt veins below the surface of the skin of a patient.

### SUMMARY

The present invention include a disruption element that is deployed from a central shaft after the shaft is plunged into the skin of a patient with a trocar like element separating the elastic layers of the skin. Once placed in the vascular bed the disruptor is navigated to the vessel for treatment. The disruptor once proximate the vessel is activated by rotation and the vessel is destroyed. Several disruption geometries are disclosed.

### BRIEF DESCRIPTION OF THE DRAWINGS

In the figures like reference numerals indicate identical structure wherein:

FIG. 1 is a perspective view of a handheld surgical tool device;

FIG. 2 is a schematic perspective view of the device treating a vein in the skin of a patient;

FIG. 3 is an illustration of a step in a method;

FIG. 4 is an illustration of a step in a method;

FIG. 5 is a phantom perspective view of the distal end of the device;

FIG. 6 is a phantom perspective view of the distal end of the device;

FIG. 7 is a phantom perspective view of the distal end of the device;

FIG. 8 is a phantom perspective view of the distal end of the device;

FIG. 9 is a phantom perspective view of the distal end of the device; and,

FIG. 10 is a phantom perspective view of the distal end of the device.

### DETAILED DESCRIPTION

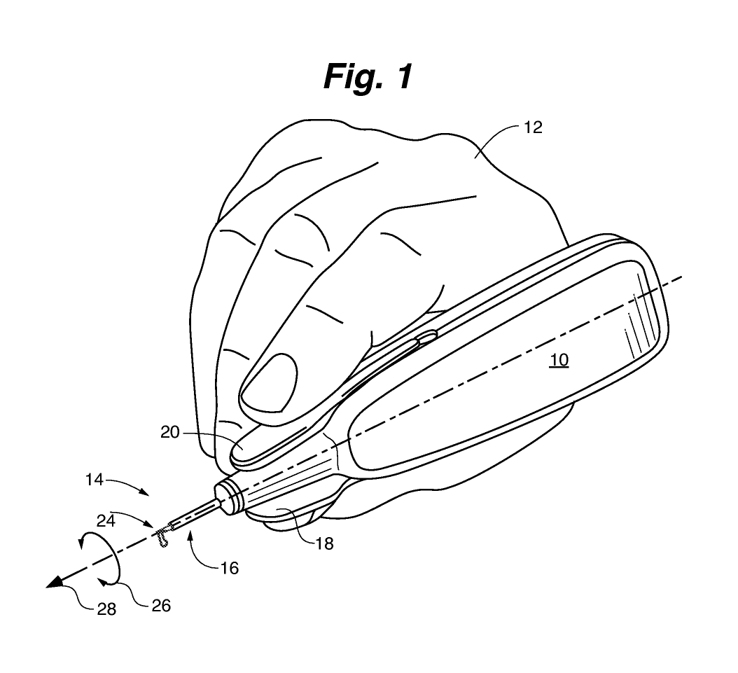

FIG. 1 shows a handheld surgical tool device 10 in the hand of a physician 12. The distal tip 14 includes a hypodermic needle 16 that is fixed with reference to the device 10. Actuation of the finger paddles 18 and 20 by the physician advances a tissue disrupting element 24 out of the hypodermic needle 16 tip and causes it to rotate in an arc 26 around an axis 28.

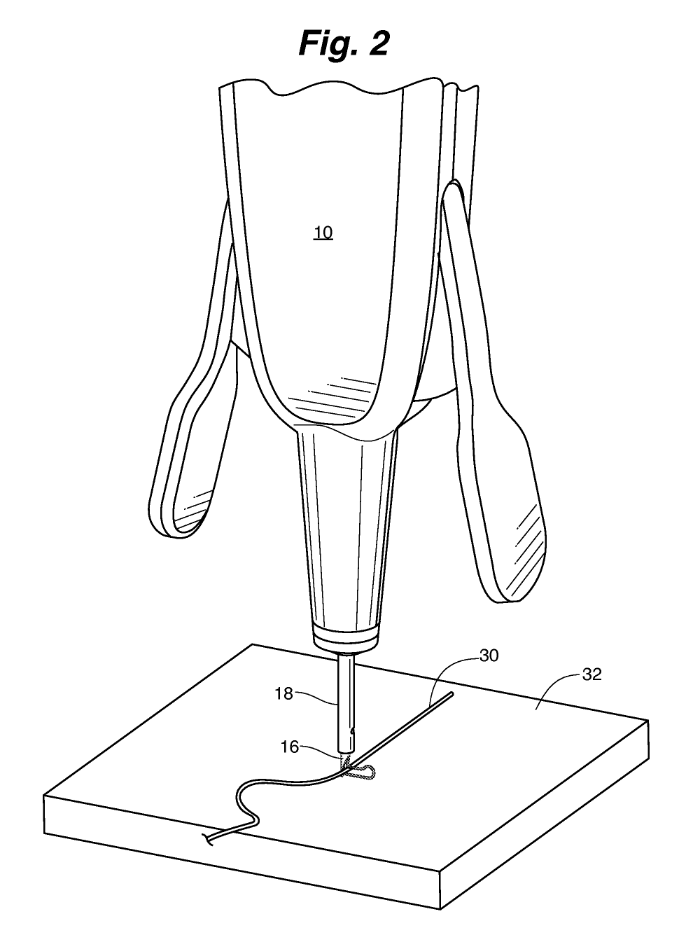

FIG. 2 shows the device 10 treating a vein 30 in the skin 32 of the patient. The physician has plunged the hypodermic needle into the skin of the patient near the vein 18 to be treated. With the distal tip proximate the vein the physician actuates the device causing the element to sweep around intercepting and destroying the vein.

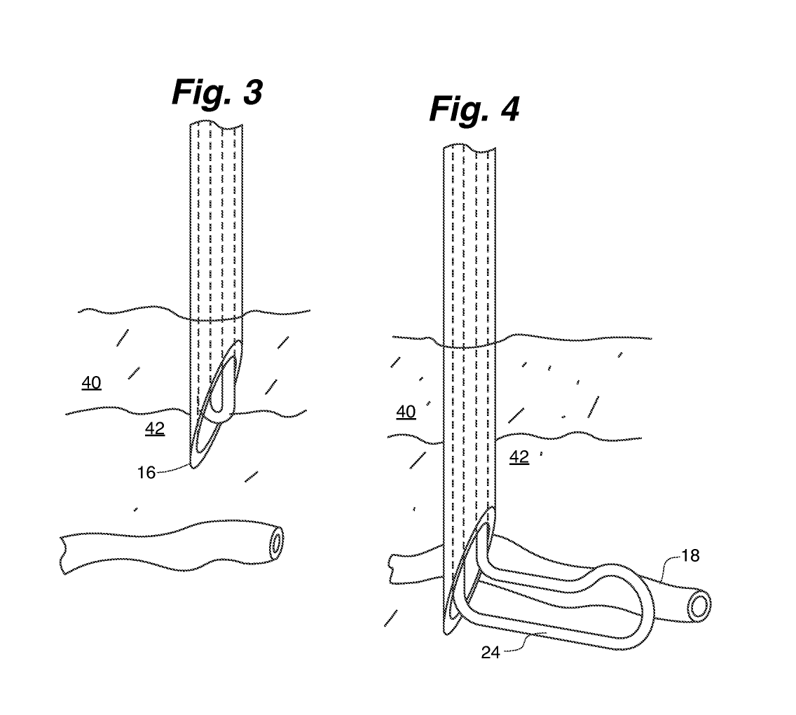

FIG. 3 and FIG. 4, considered together, illustrate steps in the disruption process. FIG. 3 shows the hypodermic needle 16 tip entering the skin and passing through the very elastic tissues 40 near the surface of the skin into the deeper dermis 42. FIG. 4 shows that needle has “missed” the vein 18 but lies proximate the vein. The physician has activated the device and a disruption loop 24 has emerged from the needle 16. Continued activation and deployed near the vein. Continued rotation of this element 24 will create a significant local destruction of the vein and nearby structures. It has been discovered by the inventors that merely cutting a vein will not obliterate it. Minor injury to the vessel is repaired. Focused localized injury is the objective. The disruption loop 24 seen in FIGS. 1-4 achieves this via blunt dissection of the tissues proximate the vein.

The remaining figures show alternative embodiments of the disruptor element.

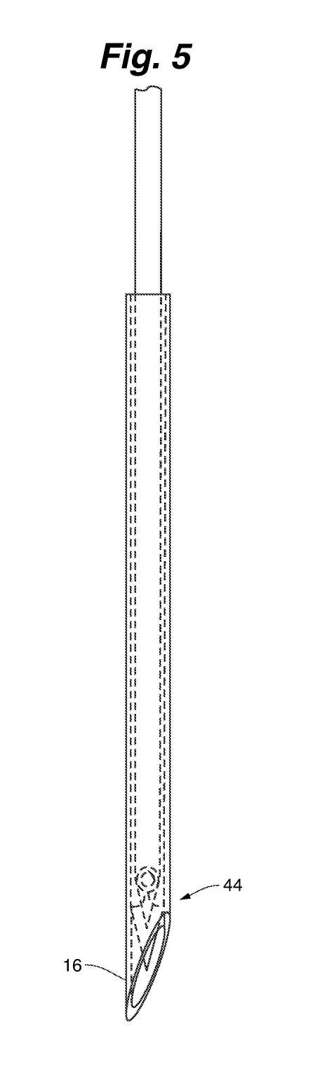

FIG. 5 shows a disruptor 44 located within the needle 16.

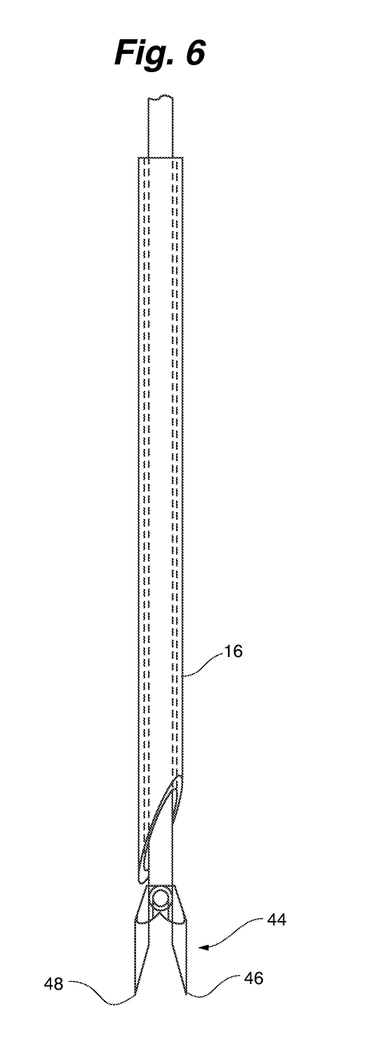

FIG. 6 shows the disruptor 44 deployed illustrating the pair of tines 48 and 46 which may be rotated to perform blunt dissection of local tissue.

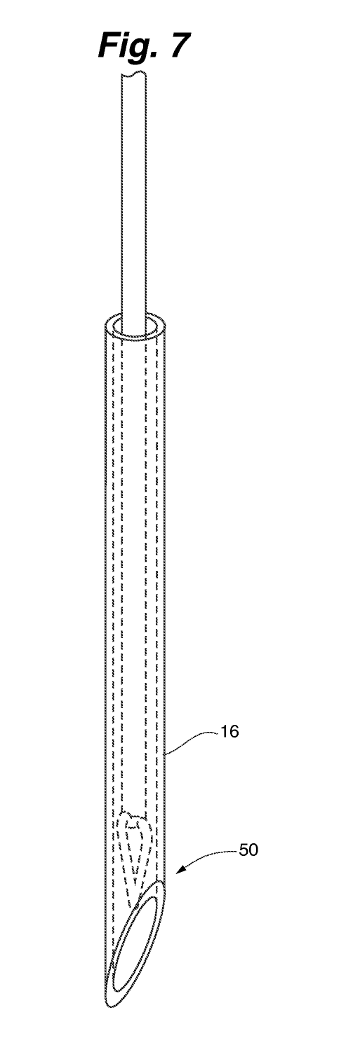

FIG. 7 shows an alternative tine disruptor structure 50 in the distal tip of the device.

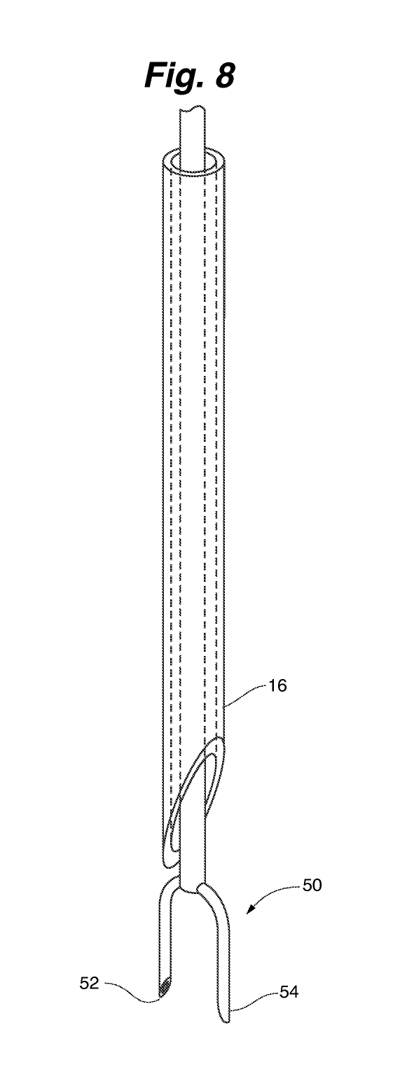

FIG. 8 shows the disruptor 50 located in a needle 16. FIG. 8 shows the disruptors deployed illustrating the pair of tines 52 and 54 which may be rotated to perform blunt dissection of local tissue.

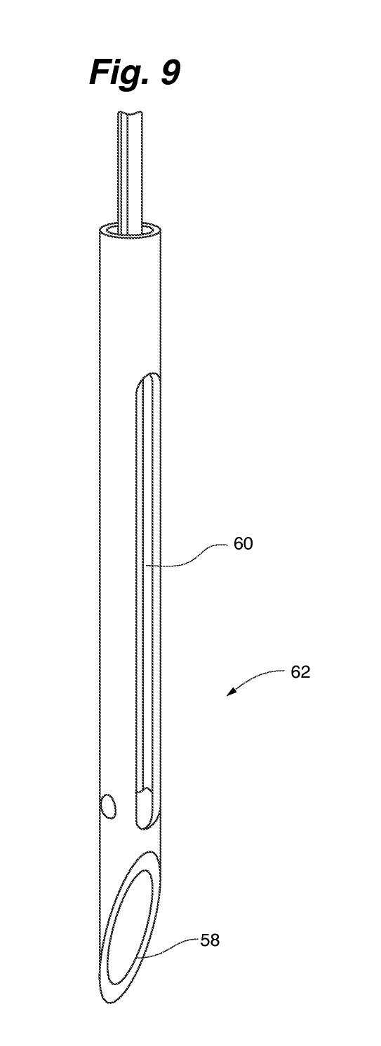

FIG. 9 shows an integrated disruptor 62 in the un-deployed state where the needle tip 58 rotates and a disruption loop 60 emerges from a lateral cut in the needle 58 which may be rotated to perform blunt dissection of local tissue.

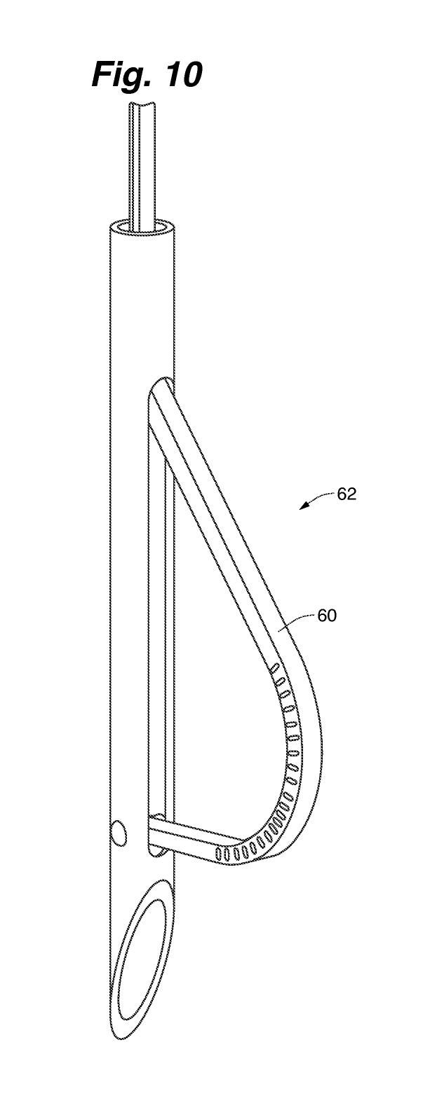

FIG. 10 shows an integrated disruptor 62 in the deployed state where the needle tip 58 rotates and a disruption loop 60 emerges from a lateral cut in the needle 58 which may be rotated to perform blunt dissection of local tissue.

What is claimed is:

1. A vein removal device comprising:
a needle for accessing tissue near the vein;
a disruptor adapted to emerge and deploy from said needle;
rotation means for rotating the disruptor, thereby providing blunt dissection of said vein.
